(19) United States
(12) Patent Application Publication
(10) Pub. No.: US 2012/0253038 A1
(43) Pub. Date: Oct. 4, 2012

(54) PROCESSES FOR THE SYNTHESIS OF BAZEDOXIFENE ACETATE AND INTERMEDIATES THEREOF

(75) Inventors: Shreerang Joshi, Thane (IN); Sachin Bhuta, Thane (IN); Sanjay Talukdar, Thane (IN); Sudhir Sawant, Thane (IN); Deepak Venkatraman, Thane (IN)

(73) Assignee: SANDOZ AG, Basel (CH)

(21) Appl. No.: 13/257,996

(22) PCT Filed: Apr. 9, 2010
(86) PCT No.: PCT/EP10/54732

§ 371 Date: Feb. 16, 2012

(30) Foreign Application Priority Data
Apr. 13, 2009 (IN) 970/MUM/2009
Jun. 12, 2009 (IN) 1419//MUM/2009

(51) Int. Cl. C07D 403/12

(52) U.S. Cl. 540/602

(57) ABSTRACT

Efficient processes for the synthesis of pharmaceutically useful compounds such as (1-[4-(2-azepan-1-yl-ethoxy)-benzyl]-2-(4-hydroxy-phenyl)-3-methyl-1H-indol-5-ol acetic acid commonly known as bazedoxifene acetate (Formula IX) using cyanomethoxybenzyl halides of Formula III, where X=Halogens e.g., Cl, F, Br, I; G=Any electron donating or electron withdrawing substituent.

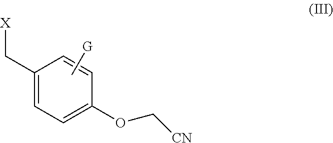

### FIELD OF THE INVENTION

The present invention provides processes for the preparation of (1-[4-(2-azepan-1-yl-ethoxy)-benzyl]-2-(4-hydroxy-phenyl)-3-methyl-1H-indol-5-ol acetic acid commonly known as Bazedoxifene acetate and related compounds from cyanomethoxybenzyl halides.

### BACKGROUND AND PRIOR ART

Cyanomethoxybenzyl halides are useful intermediates for the preparation of various pharmaceuticals. Conversion of the benzyl alcohol to corresponding benzyl halide results in the appropriate intermediate for the preparation of indole based estrogen receptors modulators as described in U.S. Pat. No. 5,998,402.

Cyanomethoxybenzyl halides are described in the following general Formula:

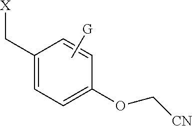

Where X=Halogens e.g., Cl, F, Br, I;
G=Any electron donating or electron withdrawing substituent.

Similarly benzylic halides that can be derived from their corresponding benzylic alcohols have been described as intermediates used in the preparation of compounds known to have inhibitory activity against various matrix metalloproteinase enzymes as well as against tumor necrosis factor α converting enzyme.

U.S. Pat. No. 6,380,166 describes process for the preparation of glucopyranosides conjugates of 2-(4-hydroxy-phenyl)-3-methyl-1-[4-(2-amin-1-yl-ethoxy)-benzyl]-1H-indol-5-ols which are useful as tissue selective estrogenic agents.

US2006/0155147 deals with the processes for the preparation of aminoethoxybenzyl alcohols. The invention provides processes and intermediates for the preparation of aminoethoxybenzyl alcohols useful in the production of pharmaceutically useful compounds.

U.S. Pat. No. 5,998,402 relates to new 2-phenyl 1-[4-(2-aminoethoxy)-benzyl] indole compounds which are useful as estrogenic agents, as well as pharmaceutical compositions and methods of treatments utilizing these compounds.

EP1025077 provides aryloxyalkyls-dialkylamines compounds useful in the production of biologically active compounds, as well as processes for their production.

Most of the published methods utilize a reducible side chain containing an ester group. The incompatibility of this side chain moiety under hydrolytic and/or reducing conditions often results in low yield of the desired product. In addition, reduction of ester functionality with lithium aluminium hydride poses potential operational problems, which would be difficult during scale up. Incompatibility of the functional groups, utilization of harsh conditions, low yield with increasing number of steps, in the prior art methods are discouraging from the commercial point of view. The present invention utilizes commercially viable synthesis of cyanomethoxybenzyl halides, which subsequently leads to cyanomethoxy, phenoxy acetic acid and amide intermediates. The present inventors have surprisingly found that the intermediates of the present invention overcome the difficulties of the prior art and may be prepared and subsequently converted to bazedoxifene acetate in high yield and purity.

### OBJECTS OF THE INVENTION

The object of the present invention is to provide an expedient commercially viable and useful process for the preparation of cyanomethoxybenzyl halides for the synthesis of pharmaceutically useful compounds.

It is another object of the present invention to provide an expedient commercially viable and useful process for the preparation of cyanomethoxy, phenoxy acetic acid and amide intermediates via the intermediate cyanomethoxybenzyl halide for the synthesis of pharmaceutically useful compounds such as bazedoxifene acetate.

It is a further object of the present invention to provide an operationally simple route of synthesis for the production of Bazedoxifene acetate in high yield and purity using the intermediate cyanomethoxybenzyl halides.

### SUMMARY OF THE INVENTION

According to an aspect of the invention there is provided a process for the synthesis of bazedoxifene acetate (Formula IX) comprising the steps of:
a. alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II);
b. converting 4-Hydroxymethyl phenoxy acetonitrile (Formula II) to 4-Chloromethyl phenoxy acetonitrile (Formula III) in presence of thionyl chloride and toluene
c. N-alkylating Formula IV with 4-Chloromethyl phenoxy acetonitrile (Formula III) in the presence of sodamide and dimethylformamide to form {4-[5-Benzyloxy-2-(4-benzyloxy-phenyl)-3-methyl-indol-1-ylmethyl]-phenoxy}-acetonitrile (Formula V);
d. reducing and debenzylating {4-[5-Benzyloxy-2-(4-benzyloxy-phenyl)-3-methyl-indol-1-yl-methyl]-phenoxy}-acetonitrile (Formula V) under catalytic hydrogenation to form 1-[4-(2-Amino-ethoxy)-benzyl]-2-(4-hydroxy-phenyl)-3-methyl-1H-indol-5-ol (Formula VI);
e. reacting 1-[4-(2-Amino-ethoxy)-benzyl]-2-(4-hydroxy-phenyl)-3-methyl-1H-indol-5-ol (Formula VI) with adipic anhydride to form 1-(2-{4-[5-Hydroxy-2-(4-hydroxy-phenyl)-3-methyl-indol-1-ylmethyl]-phenoxy}-ethyl)-azepane-2,7-dione (Formula VII);
f. reducing 1-(2-{4-[5-Hydroxy-2-(4-hydroxy-phenyl)-3-methyl-indol-1-ylmethyl]-phenoxy}-ethyl)-azepane-2,7-dione (Formula VII) with borane to give bazedoxifene free base (Formula VIII);
g. converting bazedoxifene free base (Formula VIII) to bazedoxifene acetate (Formula IX).

According to yet another aspect of the invention there is provided a process for the synthesis of bazedoxifene acetate (Formula IX) comprising the steps of:
a. alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II);
b. converting 4-Hydroxymethyl phenoxy acetonitrile (Formula II) to 4-Chloromethyl phenoxy acetonitrile (Formula III) in presence of thionyl chloride and toluene;
c. N-alkylating Formula IV with 4-Chloromethyl phenoxy acetonitrile (Formula III) in the presence of sodamide and dimethylformamide to form {-4-[5-Benzyloxy-2-(4-benzyloxy-phenyl)-3-methyl-indol-1-ylmethyl]-phenoxy}-acetonitrile (Formula V);
d. reducing and debenzylating {4-[5-Benzyloxy-2-(4-benzyloxy-phenyl)-3-methyl-indol-1-yl-methyl]-phenoxy}-acetonitrile (Formula V) under catalytic hydrogenation to form 1-[4-(2-Amino-ethoxy)-benzyl]-2-(4-hydroxy-phenyl)-3-methyl-1H-indol-5-ol (Formula VI);
e. reducing 1-[4-(2-Amino-ethoxy)-benzyl]-2-(4-hydroxy-phenyl)-3-methyl-1H-indol-5-ol (Formula VI) under catalytic hydrogenation in the presence of hexane-1,6-dial to form bazedoxifene free base (Formula VIII);
f. converting bazedoxifene free base (Formula VIII) to bazedoxifene acetate (Formula IX).

According to another aspect of the invention there is provided a process for the synthesis of bazedoxifene acetate (Formula IX) comprising the steps of:
a. alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II);
b. converting 4-Hydroxymethyl phenoxy acetonitrile (Formula II) to 4-chloromethyl phenoxy acetonitrile (Formula III) in presence of thionyl chloride and toluene;
c. N-alkylating Formula IV with 4-Chloromethyl phenoxy acetonitrile (Formula III) in the presence of sodamide and dimethylformamide to form {4-[5-Benzyloxy-2-(4-benzyloxy-phenyl)-3-methyl-indol-1-ylmethyl]-phenoxy}-acetonitrile (Formula V);
d. hydrolyzing {4-[5-benzyloxy-2-(4-benzyloxy-phenyl)-3-methyl-indol-1-ylmethyl]-phenoxy}-acetonitrile (Formula V) in the presence of sodium hydroxide at 90-100° C. to {4-[5-benzyloxy-2-(4-benzyloxy-phenyl)-3-methyl-indol-1-ylmethyl]-phenoxy}-acetic acid (Formula X);
e. reacting {4-[5-benzyloxy-2-(4-benzyloxy-phenyl)-3-methyl-indol-1-ylmethyl]-phenoxy}-acetic acid (Formula X) with 1,1′-carbonyl diimidazole and hexamethyleneimine to form 1-azepan-1-yl-2-{4-[5-benzyloxy-2-(4-benzyloxy-phenyl)-3-methyl indol-1-yl methyl]-phenoxy}-ethanone (Formula XI);
f. reducing 1-azepan-1-yl-2-{4-[5-benzyloxy-2-(4-benzyloxy-phenyl)-3-methyl-indol-1-yl methyl]-phenoxy}-ethanone (Formula XI) in the presence of borane to form 1-[4-(2-azepan-1-yl-ethoxy)-benzyl]-5-benzyloxy-2-(4-benzyloxy-phenyl)-3-methyl-1H-indole (Formula XII);
g. catalytically hydrogenating 1-[4-(2-azepan-1-yl-ethoxy)-benzyl]-5-benzyloxy-2-(4-benzyloxy-phenyl)-3-methyl-1H-indole (Formula XII) to bazedoxifene free base (Formula VIII);
h. converting bazedoxifene free base (Formula VIII) to bazedoxifene acetate (Formula IX).

According to yet another aspect of the present invention there is provided a process for the synthesis of bazedoxifene acetate (Formula IX) with high purity comprising the steps of:
a. reacting the compound of Formula XII with oxalic acid dissolved in ethanol followed by seeding crystals of the oxalate salt and filtering the reaction mixture to yield the oxalate salt of the compound of Formula XIII;
b. neutralizing the compound of Formula XIII suspended in toluene with aqueous inorganic base followed by extraction in toluene, treatment with activated charcoal and finally filtering to yield 1-[4-(2-azepan-1-yl-ethoxy)-benzyl]-5-benzyloxy-2-(4-benzyloxy-phenyl)-3-methyl-1H-indole (Formula XII) with improved purity;
c. catalytically hydrogenating 1-[4-(2-azepan-1-yl-ethoxy)-benzyl]-5-benzyloxy-2-(4-benzyloxy-phenyl)-3-methyl-1H-indole (Formula XII) in ethyl acetate to yield bazedoxifene free base (Formula VIII);
d. filtering the above reaction mixture to obtain a clear filtrate;
e. adding glacial acetic acid to the filtrate and seeding the filtrate with Bazedoxifene acetate followed by refluxing;
f. cooling of the above reaction mixture followed by filtering and washing;
g. drying the filtered product under vacuum to yield bazedoxifene acetate (Formula IX).

### DESCRIPTION OF THE ACCOMPANYING FIGURES

FIG. 1: XRD of Intermediate of Formula XIII.

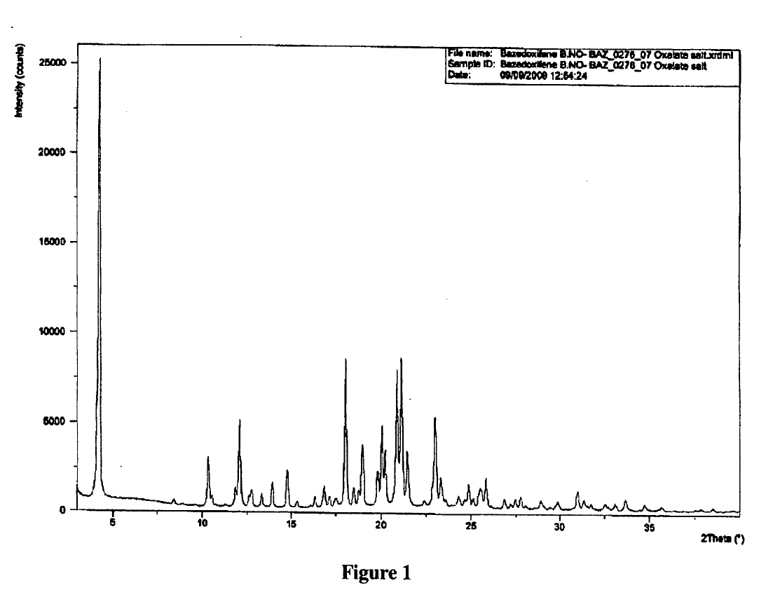

FIG. 2: IR of Intermediate of Formula XIII.

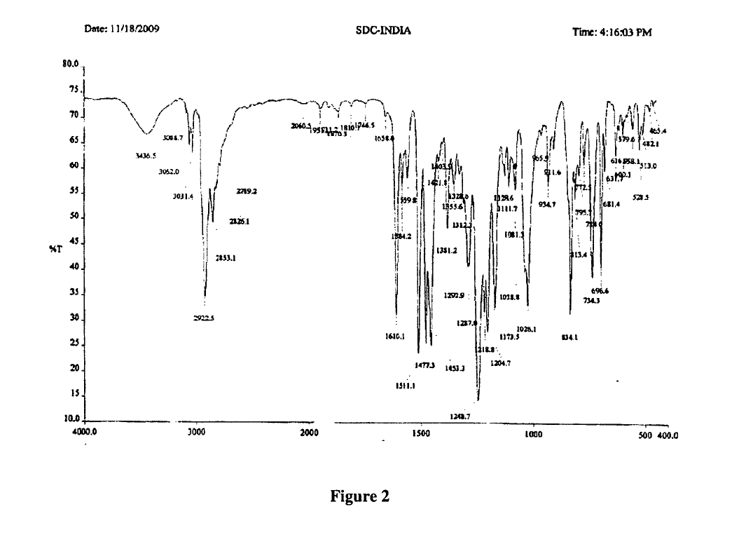

### DETAILED DESCRIPTION OF THE INVENTION WITH THE ACCOMPANYING FIGURES

The present invention provides a commercially viable synthesis of cyanomethoxybenzyl halides and it specifically describes the synthesis of 4-chloromethyl phenoxy acetonitrile. In some embodiments, the present invention provides a process for preparing a compound of Formula (III) shown below:

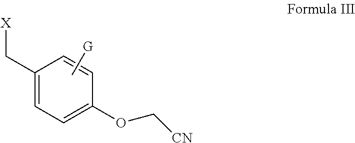

Where X=Halogens e.g., Cl, F, Br, I;
G=Any electron donating or electron withdrawing substituent.

### Representation of the Schemes

Scheme 1: Illustrates the conversion of the compound of Formula Ito a compound of Formula V via (4-Chloromethylphenoxy)acetonitrile (Formula III).
Scheme 2: Illustrates the conversion of the compound of Formula V to the compound of Formula VIII (bazedoxifene freebase) and subsequent conversion to the compound of Formula IX (bazedoxifene acetate) via two routes.
Scheme 3: Illustrates the conversion of the compound of Formula V to the compound of Formula VIII (bazedoxifene freebase) and subsequent conversion to the compound of Formula IX (bazedoxifene acetate)
Scheme 4: Illustrates the conversion of the compound of Formula XII of scheme 3 to the compound of Formula XIII followed by the conversion to the oxalate salt of the formula XIII and subsequent conversion to bazedoxifene acetate (Formula IX).

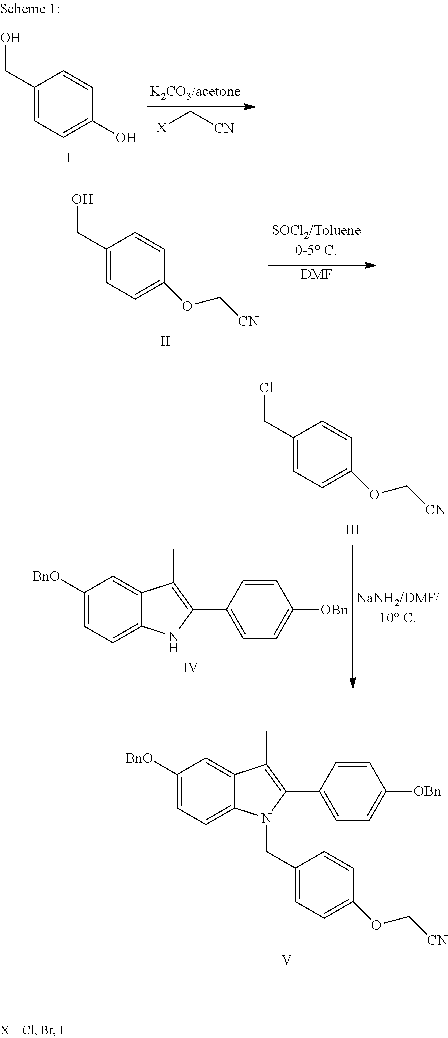

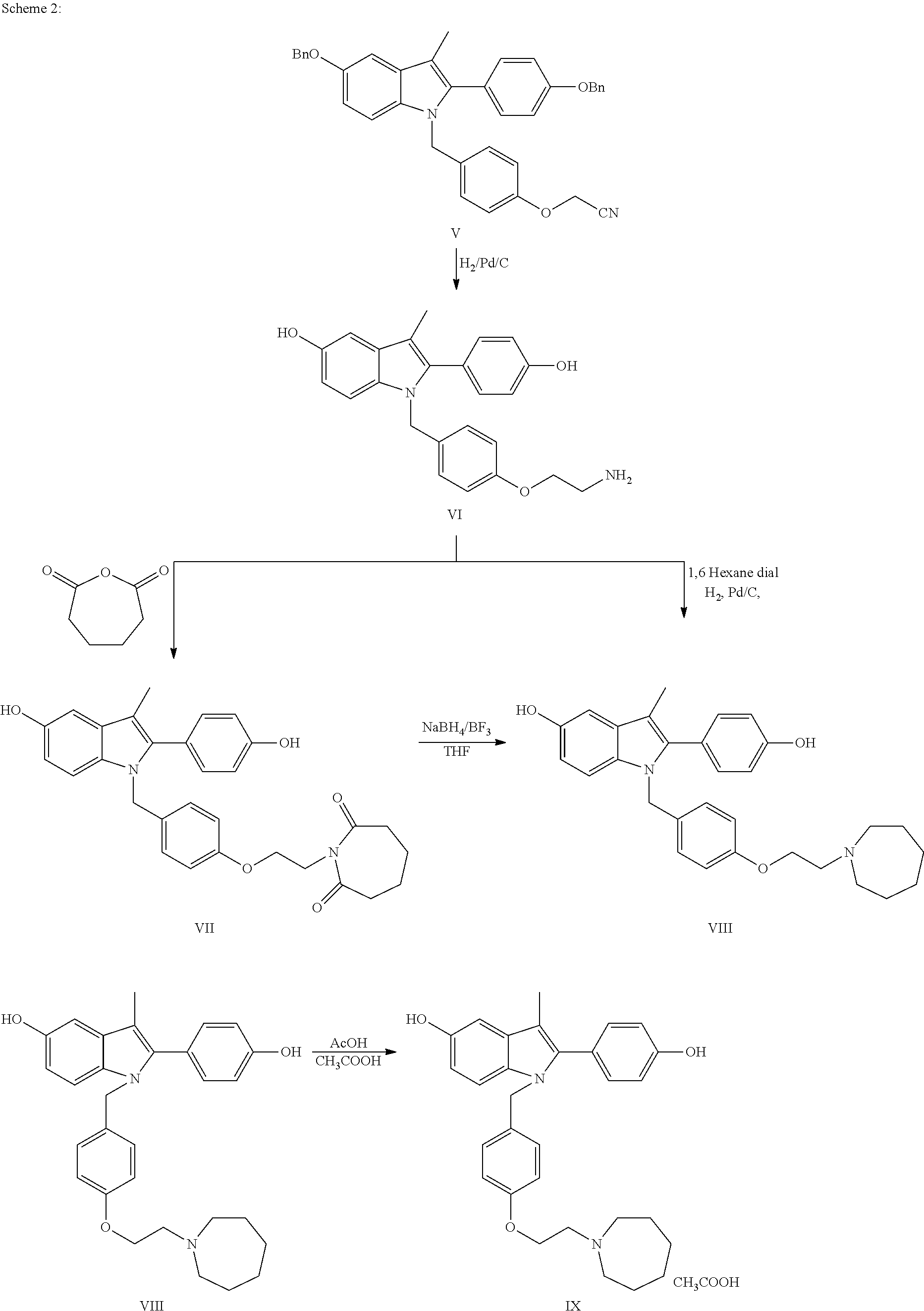

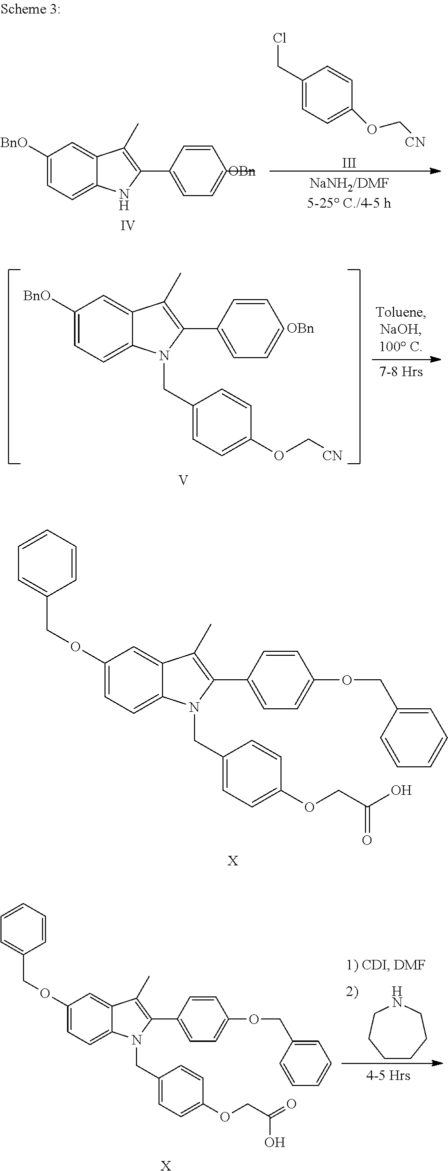

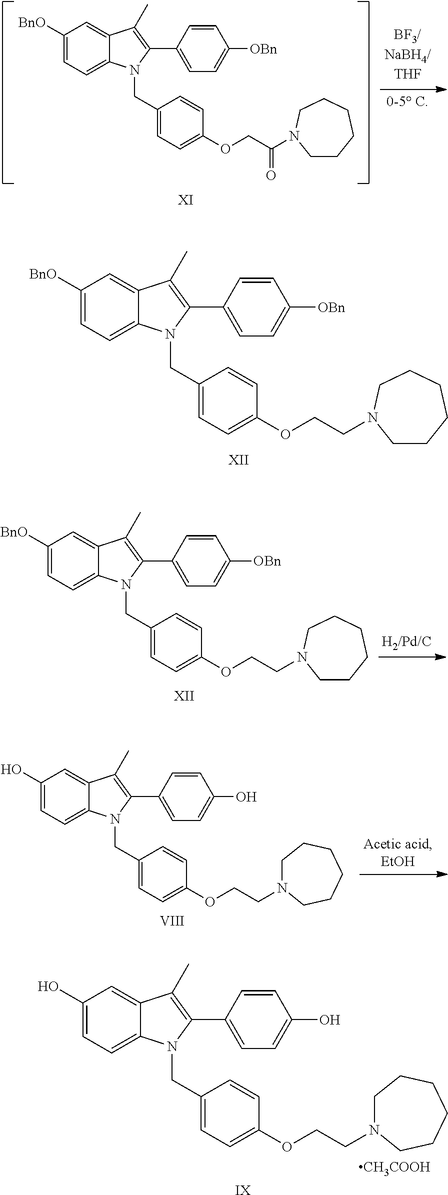

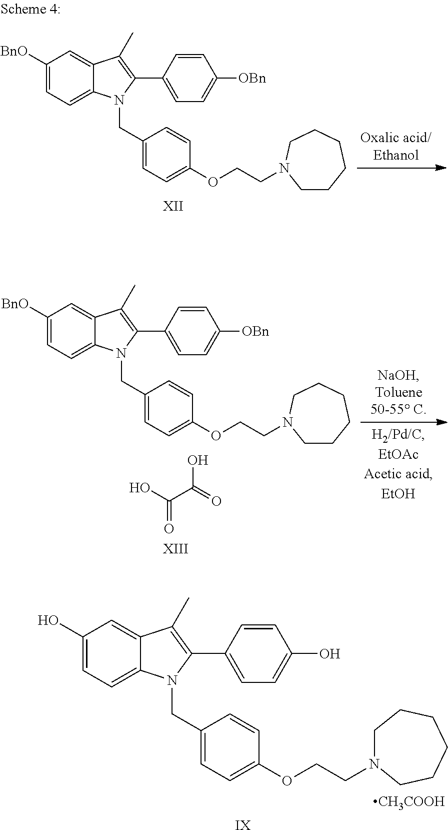

The present inventors have surprisingly found that N-Alkylation of Formula IV using Formula III leads to better purity and yield of Formula V and helps in overcoming the difficulties of the prior art and is subsequently converted to bazedoxifene acetate in high yield and purity.

According to an aspect of the present invention the process for the synthesis of bazedoxifene acetate (Formula IX) comprises alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II). Followed by converting 4-Hydroxymethyl phenoxy acetonitrile (Formula II) to 4-Chloromethyl phenoxy acetonitrile (Formula III) in presence of thionyl chloride and toluene. N-alkylating Formula IV with 4-Chloromethyl phenoxy acetonitrile (Formula III) in the presence of sodamide and dimethylformamide to form {-4-[5-Benzyloxy-2-(4-benzyloxy-phenyl)-3-methyl-indol-1-ylmethyl]-phenoxy}-acetonitrile (Formula V). [Outlined in Scheme 1]. Reducing and debenzylating {4-[5-Benzyloxy-2-(4-benzyloxy-phenyl)-3-methyl-indol-1-yl-methyl]-phenoxy}-acetonitrile (Formula V) under catalytic hydrogenation to form 1-[4-(2-Amino-ethoxy)-benzyl]-2-(4-hydroxy-phenyl)-3-methyl-1H-indol-5-ol (Formula VI). Reacting 1-[4-(2-Amino-ethoxy)-benzyl]-2-(4-hydroxy-phenyl)-3-methyl-1H-indol-5-ol (Formula VI) with adipic anhydride to form 1-(2-{4-[5-Hydroxy-2-(4-hydroxy-phenyl)-3-methyl-indol-1-ylmethyl]-phenoxy}-ethyl)-azepane-2,7-dione (Formula VII); followed by reducing 1-(2-{4-[5-Hydroxy-2-(4-hydroxy-phenyl)-3-methyl-indol-1-ylmethyl]-phenoxy}-ethyl)-azepane-2,7-dione (Formula VII) with borane to give bazedoxifene free base (Formula VIII). Finally converting bazedoxifene free base (Formula VIII) to bazedoxifene acetate (Formula IX) with acetic acid in the presence of a polar protic solvent. [Outlined in Scheme 2]

According to yet another aspect of the present invention the process for the synthesis of bazedoxifene acetate (Formula IX) comprises alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II). Followed by converting 4-Hydroxymethyl phenoxy acetonitrile (Formula II) to 4-Chloromethyl phenoxy acetonitrile (Formula III) in presence of thionyl chloride and toluene. N-alkylating Formula IV with 4-Chloromethyl phenoxy acetonitrile (Formula III) in the presence of sodamide and dimethylformamide to form {4-[5-Benzyloxy-2-(4-benzyloxy-phenyl)-3-methyl-indol-1-ylmethyl]-phenoxy}-acetonitrile (Formula V). [Outlined in Scheme 1]. Reducing and debenzylating {4-[5-Benzyloxy-2-(4-benzyloxy-phenyl)-3-methyl-indol-1-yl-methyl]-phenoxy}-acetonitrile (Formula V) under catalytic hydrogenation to form 1-[4-(2-Amino-ethoxy)-benzyl]-2-(4-hydroxy-phenyl)-3-methyl-1H-indol-5-ol (Formula VI). Reducing 1-[4-(2-Amino-ethoxy)-benzyl]-2-(4-hydroxy-phenyl)-3-methyl-1H-indol-5-ol (Formula VI) under catalytic hydrogenation in the presence of hexane-1,6-dial to form bazedoxifene free base (Formula VIII). Finally converting bazedoxifene free base (Formula VIII) to bazedoxifene acetate (Formula IX) with acetic acid in the presence of a polar protic solvent. [Outlined in Scheme 2].

Further, the present inventors identified and characterized an impurity in the synthesis of 1-[4-(2-Amino-ethoxy)-benzyl]-2-(4-hydroxy-phenyl)-3-methyl-1H-indol-5-ol (Formula VI) in a ratio of 1:1.

The impurity identified and characterized in the above process i.e. during the synthesis of the compound of Formula VI is 1-(4-Hydroxy-benzyl)-2-(4-hydroxy-phenyl)-3-methyl-1H-indol-5-ol according to the Formula XIV given below:

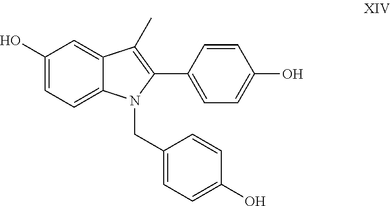

According to another aspect, the present invention also provides a process for the synthesis of Bazedoxifene acetate (Formula IX) by subjecting compound of Formula VI to column chromatography to yield Formula VI with better purity.

According to yet another aspect of the present invention the process for the synthesis of bazedoxifene acetate (Formula IX) comprises alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II). Followed by converting 4-Hydroxymethyl phenoxy acetonitrile (Formula II) to 4-chloromethyl phenoxy acetonitrile (Formula III) in presence of thionyl chloride and toluene. N-alkylating Formula IV with 4-Chloromethyl phenoxy acetonitrile (Formula III) in the presence of sodamide and dimethylformamide to form {4-[5-Benzyloxy-2-(4-benzyloxy-phenyl)-3-methyl-indol-1-ylmethyl]-phenoxy}-acetonitrile (Formula V). [Outlined in Scheme 1]. Hydrolyzing {4-[5-benzyloxy-2-(4-benzyloxy-phenyl)-3-methylindol-1-ylmethyl]-phenoxy}-acetonitrile (Formula V) in the presence of sodium hydroxide at 90-100° C. to {4-[5-benzyloxy-2-(4-benzyloxy-phenyl)-3-methyl-indol-1-ylmethyl]-phenoxy}-acetic acid (Formula X). Followed by reacting {4-[5-benzyloxy-2-(4-benzyloxy-phenyl)-3-methyl-indol-1-ylmethyl]-phenoxy}-acetic acid (Formula X) with 1,1′-carbonyl diimidazole and hexamethyleneimine to form 1-azepan-1-yl-2-{4-[5-benzyloxy-2-(4-benzyloxy-phenyl)-3-methyl indol-1-yl methyl]-phenoxy}-ethanone (Formula XI). Reducing 1-azepan-1-yl-2-{4-[5-benzyloxy-2-(4-benzyloxy-phenyl)-3-methyl-indol-1-ylmethyl]-phenoxy}-ethanone (Formula XI) with borane to form 1-[4-(2-azepan-1-yl-ethoxy)-benzyl]-5-benzyloxy-2-(4-benzyloxy-phenyl)-3-methyl-1H-indole (Formula XII). Catalytically hydrogenating 1-[4-(2-azepan-1-yl-ethoxy)-benzyl]-5-benzyloxy-2-(4-benzyloxy-phenyl)-3-methyl-1H-indole (Formula XII) to bazedoxifene free base (Formula VIII). Finally converting of bazedoxifene free base (Formula VIII) to bazedoxifene acetate (Formula IX) with acetic acid in the presence of a polar protic solvent. [Outlined in Scheme 3].

The present inventors have also surprisingly found that the purification of the intermediate 1-[4-(2-azepan-1-yl-ethoxy)-benzyl]-5-benzyloxy-2-(4-benzyloxy-phenyl)-3-methyl-1H-indole (Formula XII) of the present invention via the oxalate salt formation and subsequent crystallization yields 1-[4-(2-azepan-1-yl-ethoxy)-benzyl]-5-benzyloxy-2-(4-benzyloxy-phenyl)-3-methyl-1H-indole (Formula XII) with improved purity. The improved purity of the intermediate 1-[4-(2-azepan-1-yl-ethoxy)-benzyl]-5-benzyloxy-2-(4-benzyloxy-phenyl)-3-methyl-1H-indole (Formula XII) is important to achieve the high purity of Bazedoxifene acetate in a pharmaceutically acceptable form without the need for further purification.

Thus according to still another aspect of the present invention the process for the synthesis of a pharmaceutically acceptable form of bazedoxifene acetate with high purity comprises reacting the compound of Formula XII of the above process with oxalic acid dissolved in ethanol followed by seeding crystals of the oxalate salt and filtering the reaction mixture to yield the oxalate salt of the compound of Formula XIII. Neutralizing the compound of Formula XIII suspended in toluene with aqueous inorganic base followed by extraction in toluene, treatment with activated charcoal and finally filtering to 1-[4-(2-azepan-1-yl-ethoxy)-benzyl]-5-benzyloxy-2-(4-benzyloxy-phenyl)-3-methyl-1H-indole (Formula XII) with improved purity. Catalytic hydrogenation of 1-[4-(2-azepan-1-yl-ethoxy)-benzyl]-5-benzyloxy-2-(4-benzyloxy-phenyl)-3-methyl-1H-indole (Formula XII) in ethyl acetate to yield bazedoxifene free base (Formula VIII). Filtering the above reaction mixture to obtain a clear filtrate. To the filtrate adding glacial acetic acid followed by seeding the filtrate with Bazedoxifene acetate and then refluxing. The refluxed reaction mixture is then cooled to 25-30° C. followed by filtering and washing. Drying the filtered product under vacuum to yield the bazedoxifene acetate with high purity [Outlined in Scheme 4]. The process according to the invention as herein described yields bazedoxifene acetate with a high yield of around 90% and with >99% HPLC purity.

The polar protic solvents used in the present invention are selected from ethanol, methanol and isopropanol. The polar aprotic solvents in the present invention are selected from acetone, acetonitrile and ethyl acetate.

The aqueous inorganic base used in the present invention is selected from sodium hydroxide, potassium hydroxide, sodium bicarbonate, potassium carbonate, sodium carbonate

### Advantages of the Present Invention

1. The present route utilizes safer reaction conditions;
2. Shorter route for the synthesis of Bazedoxifene using environment friendly reagents and operational simplicity for commercial scale up;
3. It can produce bazedoxifene and related molecules at a lower cost and high purity.

While this invention has been described in terms of specific embodiments, it should be understood that the presentation is by way of illustration only and that the invention is not necessarily limited thereto. Modifications and variations within the spirit and scope of the claims that follow will be readily apparent from this disclosure, as those skilled in the art will appreciate.

The following examples further illustrate certain specific aspects and embodiments of the invention in detail and are not intended to limit the scope of the invention.

### EXAMPLES

### Example 1

### Synthesis of (4-Hydroxymethyl phenoxy)acetonitrile (Formula II)

4-Hydroxy benzyl alcohol (100 g, 0.8 mole) was dissolved in acetone (800 ml). Solid potassium carbonate (390 g, 2.8 mole) was added and stirred (15 min). Chloroacetonitrile (73 g, 0.9 moles) was added to the slurry and refluxed at 55-56° C. for 7 h (TLC, 10% MeOH in CHCl₃ absence of starting material). The slurry was filtered and filtrate concentrated to get off white solid. It was suspended in toluene (600 ml) and stirred for 1 hour. Product was then filtered and washed with toluene and dried in vacuum. wt.—118 g ; Yield: 90%.

HPLC Purity: 97.1%

¹H NMR (CDCl₃) δ 1.64 (bs, 1H), 4.63 (s, 2H), 4.77 (s, 2H), 6.99 (d, 2H, J=8 Hz), 7.34 (d, 2H, J=12 Hz) (ESI) 162(M−1)⁺.

### Example 2

### Synthesis of (4-chloromethyl phenoxy)acetonitrile (Formula III)

(4-Hydroxymethyl phenoxy)acetonitrile (Formula II) (75 g, 0.46 mole) of Example 1 was suspended in toluene (500 ml) and DMF (3.75 g). Thionyl chloride (66 ml, 0.55 moles) in toluene (150 ml) was added slowly and stirred at 0-5° C. for 2-3 h (TLC-60% EtOAc: 40% hexane-absence of starting material). Reaction was quenched with water (500 ml), layers separated and toluene layer washed with saturated sodium bicarbonate (2×200 ml) and 200 ml of distilled water. Toluene was concentrated to obtain a white solid which was suspended in n-heptane (375 ml), stirred for 30 minutes and filtered , dried in vacuum to get (4-chloromethyl phenoxy)acetonitrile (Formula III) (70 g; Yield: 84%)

HPLC Purity: 95.5%

¹H NMR (CDCl₃) δ 4.57 (s, 2H), 4.77 (s, 2H), 6.98 (d, 2H, J=8 Hz), 7.37 (d, 2H, J=12 Hz).

MS (ESI) 146.1 (M−35.5)⁺.

### Example 3

### Synthesis of {4-[5-Benzyloxy-2-(4-benzyloxy-phenyl)-3-methyl indol-1-ylmethyl]-phenoxy}-acetonitrile (Formula V)

2-substituted indole derivative (Formula IV) (80 g g, 0.19 mole) was dissolved in N,N-dimethyl formamide (DMF) (400 ml), cooled to 10-15° C. Sodamide (22.4 g, 0.57 moles) was added and stirred for 15 min. (4-chloromethyl phenoxy)acetonitrile (Formula III) of example 2 (44 g, 0.24 mole) in DMF (160 ml) was added drop wise completion, and stirred for 2-3 h at 10-15° C. (TLC, 30% EtOAc-hexane-absence of starting material). After reaction was quenched with ice-cold water (1400 ml), extracted with toluene (800 ml) and aqueous layer extracted with toluene (200 ml). Combined toluene layers were washed with saturated brine solution (2×150 ml) and Toluene was recovered under vacuum to get an off-white material. It was suspended in 800 ml Methanol and stirred for 1 hour at room temperature and filtered, washed with methanol 100 ml×2 and dried under vacuum to get an off white intermediate (Formula V). (84 g, Yield: 80%)

HPLC Purity: 95.1%

¹H NMR (CDCl₃) δ 2.24 (s, 3H), 4.70 (s, 2H), 5.10 (s, 2H), 5.12 (s, 2H), 5.13 (s, 2H), 6.82 (d, 2H, J=4 Hz), 6.90 (m, 3H), 7.03 (m, 4H), 7.14 (d, 1H, J=4 Hz), 7.21 (m, 2H), 7.22-7.50 (m, 9H).

MS (ESI) 565.5 (M+1)⁺.

### Example 4

### Purification of the {1-[4-(2-Amino-ethoxy)-benzyl]-2-(4-hydroxy-phenyl)-3-methyl-1H-indol-5-ol} (Formula VI) and Isolation of Impurity Formula XIV

Compound V of example 3 (12.0 g, 0.02 moles) was dissolved in 150 ml of 1:1 THF:Methanol was subjected to catalytic hydrogenation (Pd/C—20% wet: 50%) (5.04 g; 20 mol %) at a pressure not exceeding 3-5 kg/cm². Reaction mixture was filtered after 4 hrs (TLC—10% MeOH—CHCl₃—absence of staring material) and clear filtrate was then concentrated to obtain a pale yellow foamy solid (7.45 g, 90.8% yield). Approximately 5 gms of crude mass of Formula VI was subjected to column chromatography using silica gel and impurity of Formula XIV (2 gms) was eluted with 2% MeOH-MDC.

For Formula VI

¹H NMR (CDCl₃) δ 2.11 (s, 3H), 2.94 (t, 2H, J=4 Hz), 2.11 (s, 3H), 3.95 (t, 2H, J=4 Hz), 5.06 (s, 2H), 6.58 (d, 1H, J=4 Hz), 6.68 (m, 4H), 6.74 (m, 3H), 6.94 (d, 1H, J=8 Hz), 7.06 (dd, 2H, J=8 Hz), MS (ESI) 389(M+1)⁺.

HPLC Purity: 96.5%

For Impurity of Formula XIV

¹H NMR (CDCl₃) δ 2.09 (s, 3H), 5.05 (s, 2H), 6.55 (m, 3H), 6.64 (d, 2H, J=12 Hz), 6.79 (d, 1H, J=4 Hz), 6.84 (d, 2H, J=8 Hz), 7.15 (d, 1H, J=8 Hz), 7.17 (d, 2H, J=8 Hz), 8.70 (s, 1H), 9.25 (s, 1H), 9.67 (s, 1H).

MS (ESI) 346 (M+1)⁺.

HPLC Purity: 97%

### Example 5

### Synthesis of 1-(2-{4-[5-Hydroxy-2-(4-hydroxy-phenyl)-3-methyl-indol-1-ylmethyl]-phenoxy}-ethyl)-azepane-2,7-dione (Formula VII)

A mixture of 1-[4-(2-Amino-ethoxy)-benzyl]-2-(4-hydroxy-phenyl)-3-methyl-1H-indol-5-ol (Formula VI) of example 4, (15.1 g, 0.04 moles) and adipic anhydride (5.0 g, 0.04 moles) in toluene (200 ml) was refluxed for 5 h (TLC, 10% MeOH in CHCl₃ absence of starting material). Concentration of solvent yielded 1-(2-{4-[5-Hydroxy-2-(4-hydroxy-phenyl)-3-methyl-indol-1-ylmethyl]-phenoxy}-ethyl)-azepane-2,7-dione (Formula VII), 8.8 g.

Yield: 45.4%

### Example 6

### Synthesis of Bazedoxifene Free Base (Formula VIII)

1-(2-{4-[5-Hydroxy-2-(4-hydroxy-phenyl)-3-methyl-indol-1-ylmethyl]-phenoxy}-ethyl)-azepane-2,7-dione (Formula VII) (5 g, 0.01 moles) of example 5 in THF (10 ml) was treated with sodium borohydride (5.32 g, 0.14 moles) and boron trifluoride etherate (20 g, 0.07 moles) at 5-10° C. and stirred for 4 h (TLC, 10% MeOH in CHCl₃ absence of starting material). The reaction mixture was then heated with concentrated HCl at 50° C. for 5 h, cooled to 25-28° C. THF was removed and the reaction mixture was dissolved in toluene and neutralized using 10% NaOH solution, concentration of solvent yield bazedoxifene free base (Formula VIII) (3.8 g).

Yield: 80.7%

### Example 7

### Synthesis of Bazedoxifene Acetate (Formula IX) from Bazedoxifene Free Base (Formula VIII)

Bazedoxifene freebase, (Formula VIII) of example 6 on treatment with acetic acid in polar protic solvents produces bazedoxifene acetate.

### Example 8

### Synthesis of Bazedoxifene Free Base (Formula VIII) from 1-[4-(2-Amino-ethoxy)-benzyl]-2-(4-hydroxy-phenyl)-3-methyl-1H-indol-5-ol (Formula VI)

Hydrogenation of 1-[4-(2-Amino-ethoxy)-benzyl]-2-(4-hydroxy-phenyl)-3-methyl-1H-indol-5-ol (Formula VI) of Example 4 (3 g, 0.008 moles) in presence of hexane-1,6-dial (2 g, 0.02 moles) at 45-50° C. and 5-8 Kg/cm² pressure in presence of Pd(OH)₂ (20% on C, 50% wet, 5 g) for 6-8 h (TLC, 10% MeOH in CHCl₃ absence of starting material) yielded bazedoxifene free base (Formula VIII) (3.2 g). The free base is isolated after filtration of the reaction mixture, concentration of solvent and extraction of organic mass in ethyl acetate.

Yield: 45%

### Example 9

### Synthesis of Bazedoxifene Acetate from Bazedoxifene Free Base

Bazedoxifene freebase (Formula VIII) of example 8 on treatment with acetic acid in polar protic solvents produces bazedoxifene acetate.

### Example 10

### Hydrolysis of {4-[5-Benzyloxy-2-(4-benzyloxy-phenyl)-3-methyl-indol-1-ylmethyl]phenoxy}-acetonitrile (V) to {4-[5-Benzyloxy-2-(4-benzyloxy-phenyl)-3-methyl-indol-1-ylmethyl]-phenoxy}-acetic acid (Formula X)

Formula V of example 3 was dissolved in 160 ml Toluene and heated to 90-100° C. 26% NaOH solution (90 g in 250 ml water) was added and maintained for a period of 8 h or until completion. (TLC—30% ethylacetate:hexane—absence of starting material)—Reaction mass was then quenched with water (250 ml) and filtered , suspended in ethyl acetate (700 ml) and water (200 ml) and acidified with 3 N HCl (until pH 1.6). Ethyl acetate was washed with 150 ml saturated brine solution, dried and concentrated to obtain a crude solid which was suspended in methanol (600 ml) heated to 50-55° C. for 1 hr , cooled to 25-30° C. and filtered , washed with methanol (100 ml×2) and dried completely to get 4-[5-Benzyloxy-2-(4-benzyloxy-phenyl)-3-methyl-indol-1-ylmethyl]-phenoxy}-acetic acid

(Formula X) (75 g; Yield: 85%)

HPLC Purity: 98.5%

¹H NMR (CDCl₃) δ 2.20 (s, 3H), 4.57 (s, 2H), 5.08 (s, 2H), 5.11 (s, 2H), 5.12 (s, 2H), 6.74 (d, 2H, J=8 Hz), 6.87 (m, 3H), 7.03 (m, 4H), 7.14 (d, 1H, J=4 Hz), 7.26 (d, 2H, J=8 Hz), 7.31-7.49 (m, 9H). MS (ESI) 584(M+1)⁺.

### Example 11

### Synthesis of 1-Azepan-1-yl-2-{4-[5-benzyloxy-2-(4-benzyloxy-phenyl)-3-methyl indol-1-yl methyl]-phenoxy}-ethanone (Formula XI)

Formula X of example 10 (48.0 g, 0.08 mole) was dissolved in DMF (250 ml) and 1,1′-carbonyl diimidazole (20.0 g, 0.12 mole) was added. The reaction mixture was stirred at 25-30° C. for 3 hours. A solution of hexamethyleneimine (16.32 g, 0.16 mole) in DMF (30 ml) was added drop wise over 30-45 minutes and stirred. On completion, (TLC; 10% MeOH/CHCl₃), water (500 ml) was added to reaction mixture and product extracted in toluene (300 ml) after adjusting the pH to 5-6 with 3N HCl (75 ml). Aqueous layer was re-extracted with toluene (100 ml×2), combined toluene layer was washed with saturated brine solution (100 ml), dried , concentrated to half the volume and treated with silica (10 g, 60-300 mesh size) and activated charcoal (10 g) treatment , filtered and concentrated to get an off white solid

Formula XI, (53.6 g, Yield: 98%)

HPLC Purity: 99.3%

¹H NMR (CDCl₃) δ 1.53-1.55 (m, 4H), 1.60 (m, 4H), 2.23 (s, 3H), 3.47 (t, 2H, J=8 Hz), 3.53 (t, 2H, J=8 Hz), 4.62 (s, 2H), 5.10 (s, 2H), 5.11 (s, 2H), 5.11 (s, 2H), 5.13 (s, 2H), 6.83 (d, 2H, J=8 Hz), 6.87 (m, 3H), 7.04 (m, 5H), 7.13 (d, 1H, J=4 Hz), 7.24 (d, 2H, J=8 Hz), 7.32-7.50 (m, 8H). MS (ESI) 665.4 (M+1)⁺.

### Example 12

### Reduction of 1-Azepan-1-yl-2-{4-[5-benzyloxy-2-(4-benzyloxy-phenyl)-3-methyl indol-1-yl methyl]-phenoxy}-ethanone (Formula XI) to 1-[4-(2-Azepan-1-yl-ethoxy)-benzyl]-5-benzyloxy-2-(4-benzyloxy-phenyl)-3-methyl-1H-indole (Formula XII)

NaBH₄ (24 g, 0.036 mole) was added to solution of amide intermediate Formula XI of example 11 (60 g in 900 ml THF) and the reaction mixture was cooled to 10-15° C. To this BF₃.etherate (80 ml, 0.315 moles) was added drop wise over 30-40 minutes. Reaction mixture was then heated to 50° C. for 3 hrs (TLC—60% ethylacetate:hexane—absence of Formula XI) and then quenched with 900 ml water at 25-30° C., acidified using 210 ml conc. HCl. After refluxing at 60-65° C. for 6 hours, (TLC—60% ethyl acetate:hexane—absence of intermediate) the mixture was cooled to 40° C. and THF was recovered on rotavapor, suspension filtered and wet cake was suspended in toluene (500 ml) stirred for 1 hour at 25-30° C., filtered , washed with toluene (3×60 ml) and was dried under suction. The HCl salt of Formula XII, so obtained was neutralized with 35% NaOH (100 ml), water (50 ml) and simultaneously extracted with toluene (400 ml). Toluene layer was then washed with saturated brine solution (100 ml), concentrated to half the volume and treated with silica and charcoal and filtered through celite bed and concentrated to get an off-white solid (Formula XII). (36 g, Yield: 70%).

HPLC Purity: 98.1%

¹H NMR (CDCl₃) δ 1.60-1.66 (m, 8H), 2.25 (s, 3H), 2.78 (t, 4H, J=8 Hz), 2.94 (t, 2H, J=8 Hz), 4.02 (t, 2H, J=8 Hz), 5.10 (s, 2H), 5.11 (s, 2H), 5.12 (s, 2H), 5.14 (s, 2H), 6.75 (d, 2H, J=8 Hz), 6.88 (m, 3H), 7.03 (m, 5H), 7.05 (d, 2H, J=8 Hz), 7.12-7.43 (m, 1H)⁺.

MS (ESI) 651.4 (M+1)⁺.

### Example 13

### Debenzylation of 1-[4-(2-Azepan-1-yl-ethoxy)-benzyl]-5-benzyloxy-2-(4-benzyloxy-phenyl)-3-methyl-1H-indole (Formula XII) to Bazedoxifene free base (Formula VIII)

To a solution of intermediate of Formula XII of example 12 (1.0 g, 0.002 mole) in acetone (20 ml) was added ammonium formate (0.5 g, 0.008 mole) followed by Pd(OH)₂/C (0.86 g, 10%, 50% wet). The reaction mixture was refluxed. On completion (TLC, 10% MeOH/CHCl₃), catalyst was filtered through Celite bed and acetone was removed to get concentrated mass, which was dissolved in ethyl acetate and washed with water. Ethyl acetate layer dried over anhydrous sodium sulphate and concentrated to get an off-white foam of Formula VIII.

Yield: 0.6 g (85% yield).

### Example 14

### Synthesis of Bazedoxifene Acetate (Formula IX) from Bazedoxifene Free Base (Formula VIII)

Bazedoxifene free base (Formula VIII) of example 13 on treatment with acetic acid in polar protic solvents produces bazedoxifene acetate.

### Example 15

### Purification of Formula XII by Oxalate Salt (Formula XIII) Formation

Crude solid of Formula XII of example 12 (21 g, 0.032 moles) was dissolved in 140 ml Toluene and heated to 55-60° C. Oxalic acid (3.8 g, 0.03 moles) was dissolved in 25 ml Ethanol and added over 5-10 minutes. Seed crystals of Oxalate salt were added and reaction mixture stirred for 3 hours. Reaction mixture was cooled to 25-30° C. and off white solid was filtered on Buchner funnel and washed with Toluene 25 ml×2 times and then dried in vacuum.(23.6 g Yield: 95%)

HPLC Purity: 99.7%

### Example 16

### Preparation of Bazedoxifene Acetate API (Formula IX)

Oxalate salt Formula XIII (14.4. g, 0.018 moles) of example 15 was suspended in 100 ml Toluene and heated to 55-60° C. NaOH (3 g, 0.075 moles) was dissolved in 30 ml distilled water and added to reaction flask and stirred for 1.5 hours. Reaction mixture cooled to 25-30° C. and layers separated. Aqueous layer extracted with Toluene 50 ml. Combined Toluene layer washed with distilled water 50 ml×2 and treated with activated charcoal for 15-20 minutes. Charcoal was filtered through celite bed and clear filtrate was concentrated to get Benzylated Bazedoxifene as an off-white solid (Formula XII). (Wt.12.1 g)

Above off white solid (Formula XII) (12.1 g, 0.018 moles) was dissolved in Ethyl acetate (100 ml) and hydrogenated using 5% Pd/C (dry 1 gm) for 1.5 to 2 hours. Reaction progress was monitored by TLC and after completion; reaction mixture was filtered through celite bed. To clear filtrate glacial acetic acid (2.1 ml, 0.036 moles) and seed crystals of Bazedoxifene acetate were added and heated to reflux for about 3 hours. Reaction mixture was cooled to 25-30° C. and white product was filtered and washed with Ethyl acetate 50 ml×2 times and dried under vacuum to get Bazedoxifene acetate API.(8 g, Yield: 80%).

HPLC Purity: 99.7%

¹H NMR (CDCl₃) δ 1.45-1.55 (m, 8H), 1.89 (s, 3H), 2.08 (s, 3H), 2.63 (t, 4H, J=6 Hz), 2.77 (t, 2H, J=6 Hz), 3.90 (t, 2H, J=6 Hz), 5.08 (s, 2H), 6.55 (dd, 1H, J=2 Hz), 6.72 (m, 4H), 6.78 (d, 1H, J=2 Hz), 6.83 (d, 2H, J=8.4 Hz), 7.04 (d, 1H, J=8.8 Hz), 7.14 (d, 2H, J=8.4 Hz).

MS (ESI) 471(M+1)⁺.

1. A process for the synthesis of bazedoxifene acetate (Formula IX) comprising the steps of:
a. alkylating phenolic hydroxyl of 4-hydroxybenzyl alcohol (Formula I) with chloroacetonitrile in presence of potassium carbonate and acetone to form 4-hydroxymethyl-phenoxy acetonitrile (Formula II);
b. converting 4-Hydroxymethyl phenoxy acetonitrile (Formula II) to 4-chloromethyl phenoxy acetonitrile (Formula III) in presence of thionyl chloride and toluene;
c. N-alkylating Formula IV with 4-Chloromethyl phenoxy acetonitrile (Formula III) in the presence of sodamide and dimethylformamide to form {4-[5-Benzyloxy-2-(4-benzyloxy-phenyl)-3-methyl-indol-1-ylmethyl]-phenoxy}-acetonitrile (Formula V);
d. hydrolyzing {4-[5-benzyloxy-2-(4-benzyloxy-phenyl)-3-methyl indol-1-ylmethyl]-phenoxy}-acetonitrile (Formula V) in the presence of sodium hydroxide at 90-100° C. to {4-[5-benzyloxy-2-(4-benzyloxy-phenyl)-3-methyl-indol-1-ylmethyl]-phenoxy}-acetic acid (Formula X);
e. reacting {4-[5-benzyloxy-2-(4-benzyloxy-phenyl)-3-methyl-indol-1-ylmethyl]-phenoxy}-acetic acid (Formula X) with 1,1′-carbonyl diimidazole and hexamethyleneimine to form 1-azepan-1-yl-2-{4-[5-benzyloxy-2-(4-benzyloxy-phenyl)-3-methyl indol-1-yl methyl]-phenoxy}-ethanone (Formula XI);
f. reducing 1-azepan-1-yl-2-{4-[5-benzyloxy-2-(4-benzyloxy-phenyl)-3-methyl indol-1-yl methyl]-phenoxy}-ethanone (Formula XI) in the presence of borane to form I-[4-(2-azepan-1-yl-ethoxy)-benzyl]-5-benzyloxy-2-(4-benzyloxy-phenyl)-3-methyl-1H-indole (Formula XII);
g. catalytically hydrogenating I-[4-(2-azepan-1-yl-ethoxy)-benzyl]-5-benzyloxy-2-(4-benzyloxy-phenyl)-3-methyl-1H-indole (Formula XII) to bazedoxifene free base (Formula VIII); and
h. converting bazedoxifene free base (Formula VIII) to bazedoxifene acetate (Formula IX).

2. The process according to claim 1, further comprising the steps of:
a. reacting the compound of Formula XII with oxalic acid dissolved in ethanol followed by seeding crystals of the oxalate salt and filtering the reaction mixture to yield the oxalate salt of the compound of Formula XIII;
b. neutralizing the compound of Formula XIII suspended in toluene with aqueous inorganic base followed by extracting in toluene, treating with activated charcoal and finally filtering to yield I-[4-(2-azepan-1-yl-ethoxy)-benzyl]-5-benzyloxy-2-(4-benzyloxy-phenyl)-3-methyl-1H-indole (Formula XII) with improved purity;
c. catalytically hydrogenating I-[4-(2-azepan-1-yl-ethoxy)-benzyl]-5-benzyloxy-2-(4-benzyloxy-phenyl)-3-methyl-1H-indole (Formula XII) in ethyl acetate to yield bazedoxifene free base (Formula VIII);
d. filtering the above reaction mixture to obtain a clear filtrate;
e. adding glacial acetic acid to the filtrate and seeding the filtrate with Bazedoxifene acetate followed by refluxing;
f. cooling of the above reaction mixture followed by filtering and washing; and
g. drying the filtered product under vacuum to yield the bazedoxifene acetate (Formula IX).

3. The process according to claim 2, wherein the aqueous inorganic base is selected from the group comprising of sodium hydroxide, sodium bicarbonate, potassium carbonate, sodium carbonate, potassium hydroxide.

4. The process of preparing Bazedoxifene acetate (formula IX) also comprising:
a) N-alkylating Formula IV with 4-Chloromethyl phenoxy acetonitrile (Formula III) in the presence of sodamide and dimethylformamide to form {4-[5-Benzyloxy-2-(4-benzyloxy-phenyl)-3-methyl-indol-1-ylmethyl]-phenoxy}-acetonitrile (Formula V); and
b) reducing and debenzylating {4-[5-Benzyloxy-2-(4-benzyloxy-phenyl)-3-methyl-indol-I-yl-methyl]-phenoxy}-acetonitrile (Formula V) under catalytic hydro genati on to form 1-[4-(2-Amino-ethoxy)-benzyl]-2-(4-hydroxy-phenyl)-3-methyl-1H-indol-5-ol (Formula VI).

5. The process for the synthesis of bazedoxifene acetate (Formula IX) with improved purity according to claim 1, wherein the compound of Formula VI is further subjected to column chromatography.

6. The process according to claim 1, wherein the conversion of bazedoxifene free base (Formula VIII) to bazedoxifene acetate (Formula IX) is carried out in the presence of polar protic solvents.

7. The process according to claim 6 wherein the polar protic solvent is selected from methanol, ethanol, isopropanol.

8. The process according to claim 1, wherein the catalytic hydrogenation is effected in the presence of palladium on carbon or palladium hydroxide on carbon.
